(19) United States
(12) Patent Application Publication
(10) Pub. No.: US 2007/0004781 A1
(43) Pub. Date: Jan. 4, 2007

(54) Crystalline N-(2-((4-hydroxyphenyl)amino)pyridin-3-yl)-4-methoxybenzenesulfonamide hydrochloride

(76) Inventors: Eric A. Schmitt, Libertyville, IL (US); Ira S. Buckner, Iowa City, IA (US); Geoff G.Z. Zhang, Libertyville, IL (US); Roger F. Henry, Wildwood, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/332,015

(22) Filed: Jan. 13, 2006

Related U.S. Application Data
(60) Provisional application No. 60/643,571, filed on Jan. 13, 2005.

(51) Int. Cl. A61K 31/44; C07D 213/72

(52) U.S. Cl. 514/352; 546/304

(57) ABSTRACT

Crystalline N-(2-((4-hydroxyphenyl)amino)pyridin-3-yl)-4-methoxybenzenesulfonamide hydrochloride, ways to make it, compositions containing it and methods of treatment of diseases using it are disclosed.

This application claims priority to co-pending U.S. Provisional Application Ser. No. 60/643,571, filed Jan. 13, 2005.

### FIELD OF THE INVENTION

This invention pertains to crystalline N-(2-((4-hydroxyphenyl)amino)pyridin-3-yl)-4-methoxybenzenesulfonamide hydrochloride, ways to make it, compositions containing it and methods of treatment of diseases using it.

### BACKGROUND OF THE INVENTION

Crystallinity of compounds may effect, among other physical and mechanical properties, their solubility, dissolution rate, hardness, compressibility and melting point. There is therefore an existing need in the chemical and therapeutic arts for identification of novel crystalline forms of salts of drugs and ways of reproducibly making them.

### SUMMARY OF THE INVENTION

One embodiment of this invention pertains to crystalline N-(2-((4-hydroxyphenyl)amino)pyridin-3-yl)-4-methoxybenzenesulfonamide hydrochloride.

Another embodiment pertains to N-(2-((4-Hydroxyphenyl)amino)pyridin-3-yl)-4-methoxybenzenesulfonamide hydrochloride Crystalline Form 1 characterized, when measured at about 25° C. with Cu-Kα radiation, by a powder diffraction pattern with at least three peaks having respective 2θ values of about 11.7°, 12.8°, 13.2°, 14.4°, 15.9°, 16.7°, 17.2°, 19.2°, 21.1°, 21.4°, 22.7°, 22.9°, 24.2°, 24.7°, 25.5°, 25.6° or 26.0°.

Still another embodiment pertains to N-(2-((4-Hydroxyphenyl)amino)pyridin-3-yl)-4-methoxybenzenesulfonamide hydrochloride Crystalline Form 1 characterized, when measured at about 25° C. with Mo-Kα radiation, by respective lattice parameters a, b and c of about 8.4918 Å±0.0009 Å, 16.389 Å±0.0004 Å and 13.358 Å±0.0002 Å, and β of 98.052°.

Still another embodiment pertains to crystalline N-(2-((4-hydroxyphenyl)amino)pyridin-3-yl)-4-methoxybenzenesulfonamide hydrochloride having substantial crystalline purity.

Still another embodiment pertains to N-(2-((4-Hydroxyphenyl)amino)pyridin-3-yl)-4-methoxybenzenesulfonamide hydrochloride Crystalline Form 1 having substantial crystalline purity and characterized, when measured at about 25° C. with Cu-Kα radiation, by a powder diffraction pattern with at least three peaks having respective 2θvalues of about 11.7°, 12.8°, 13.2°, 14.4°, 15.9°, 16.7°, 17.2°, 19.2°, 21.1°, 21.4°, 22.7°, 22.9°, 24.2°, 24.7°, 25.5°, 25.6° or 26.0°.

Still another embodiment pertains to N-(2-((4-Hydroxyphenyl)amino)pyridin-3-yl)-4-methoxybenzenesulfonamide hydrochloride Crystalline Form 1 having substantial crystalline purity and characterized, when measured at about 25° C. with Mo-Kα radiation, by respective lattice parameters a, b and c of about 8.4918 Å±0.0009 Å, 16.389 Å±0.0004 Å and 13.358 Å±0.0002 Å, and β of 98.052°.

Still another embodiment pertains to crystalline N-(2-((4-hydroxyphenyl)amino)pyridin-3-yl)-4-methoxybenzenesulfonamide hydrochloride having substantial crystalline purity and substantial chemical purity.

Still another embodiment pertains to N-(2-((4-Hydroxyphenyl)amino)pyridin-3-yl)-4-methoxybenzenesulfonamide hydrochloride Crystalline Form 1 having substantial crystalline purity and substantial chemical purity and characterized, when measured at about 25° C. with Cu-Kα radiation, by a powder diffraction pattern with at least three peaks having respective 2θvalues of about 11.7°, 12.8°, 13.2°, 14.4°, 15.9°, 16.7°, 17.2°, 19.2°, 21.1°, 21.4°, 22.7°, 22.9°, 24.2°, 24.7°, 25.5°, 25.6° or 26.0°.

Still another embodiment pertains to N-(2-((4-Hydroxyphenyl)amino)pyridin-3-yl)-4-methoxybenzenesulfonamide hydrochloride Crystalline Form 1 having substantial crystalline purity and substantial chemical purity and characterized, when measured at about 25° C. with Mo-Kα radiation, by respective lattice parameters a, b and c of about 8.4918 Å±0.0009 Å, 16.389 Å±0.0004 Å and 13.358 Å±0.0002 Å, and β of 98.052°.

Still another embodiment pertains to compositions comprising an excipient and a therapeutically acceptable amount of a crystalline N-(2-((4-hydroxyphenyl)amino)pyridin-3-yl)-4-methoxybenzenesulfonamide hydrochloride.

Still another embodiment pertains to methods for treating cancer in a mammal comprising administering a therapeutically effective amount of a crystalline N-(2-((4-hydroxyphenyl)amino)pyridin-3-yl)-4-methoxybenzenesulfonamide hydrochloride.

Still another embodiment pertains to processes for making crystalline N-(2-((4-Hydroxyphenyl)amino)pyridin-3-yl)-4-methoxybenzenesulfonamide hydrochloride Crystalline Form 1,
said processes comprising:

providing a mixture comprising N-(2-((4-hydroxyphenyl)amino)pyridin-3-yl)-4-methoxybenzenesulfonamide hydrochloride and solvent, wherein said N-(2-((4-hydroxyphenyl)amino)pyridin-3-yl)-4-methoxybenzenesulfonamide hydrochloride is completely soluble in said solvent;

causing N-(2-((4-Hydroxyphenyl)amino)pyridin-3-yl)-4-methoxybenzenesulfonamide hydrochloride Crystalline Form 1 to exist in said mixture, said N-(2-((4-Hydroxyphenyl)amino)pyridin-3-yl)-4-methoxybenzenesulfonamide hydrochloride Crystalline Form 1, when isolated and measured at about 25° C. with Cu-Kα radiation, characterized by a powder diffraction pattern with at least three peaks having respective 2θvalues ofabout 11.7°, 12.8°, 13.2°, 14.4°, 15.9°, 16.7°, 17.2°, 19.2°, 21.1°, 21.4°, 22.7°, 22.9°, 24.2°, 24.7°, 25.5°, 25.6° or 26.0°; and

isolating said crystalline N-(2-((4-Hydroxyphenyl)amino)pyridin-3-yl)-4-methoxybenzenesulfonamide hydrochloride Crystalline Form 1.

Still another embodiment pertains to processes for making N-(2-((4-Hydroxyphenyl)amino)pyridin-3-yl)-4-methoxybenzenesulfonamide hydrochloride Crystalline Form 1,
said processes comprising:

providing a mixture comprising N-(2-((4-hydroxyphenyl)amino)pyridin-3-yl)-4-methoxybenzenesulfonamide hydrochloride and solvent, wherein said N-(2-((4-hydroxyphenyl)amino)pyridin-3-yl)-4-methoxybenzenesulfonamide hydrochloride is completely soluble in said solvent;

causing crystalline N-(2-((4-Hydroxyphenyl)amino)pyridin-3-yl)-4-methoxybenzenesulfonamide hydrochloride Crystalline Form 1 to exist in said mixture, said N-(2-((4-hydroxyphenyl)amino)pyridin-3-yl)-4-methoxybenzenesulfonamide hydrochloride, when isolated and measured at about 25° with Mo-Kα radiation, characterized by respective lattice parameters a, b and c of about a, b and c of about 8.4918 Å±0.0009 Å, 16.389 Å±0.0004 Å and 13.358 Å±0.0002 Å, and β of 98.052°; and

isolating said N-(2-((4-Hydroxyphenyl)amino)pyridin-3-yl)-4-methoxybenzenesulfonamide hydrochloride Crystalline Form 1.

### DETAILED DESCRIPTION OF THE INVENTION

This invention pertains to crystalline N-(2-((4-hydroxyphenyl)amino)pyridin-3-yl)-4-methoxybenzenesulfonamide hydrochloride, a particular example of which is N-(2-((4-Hydroxyphenyl)amino)pyridin-3-yl)-4-methoxybenzenesulfonamide hydrochloride Crystalline Form 1 which may be characterized as described hereinabove.

The term “crystalline,” as used herein, means having a regularly repeating arrangement of molecules or external face planes.

Moieties herein are represented by capital letters with numerical superscripts and are specifically embodied. For example, 4-methoxyphenyl and (2Z) ((4-hydroxyphenyl)amino)-pyridin-3-yl may be represented by R¹ and R², respectively.

The term “substantial crystalline purity,” as used herein, means at least about 95% crystalline purity, preferably about 97% crystalline purity, more preferably about 99% crystalline purity, and most preferably about 100% crystalline purity.

The term “crystalline purity,” as used herein, means percentage of a particular crystalline R¹SO₂NHR².HCl in a sample which may contain amorphous R¹SO₂NHR², one or more than one other crystalline forms of R¹SO₂NHR².HCl, or a mixture thereof.

The term “substantial chemical purity,” as used herein, means about 95% chemical purity, preferably about 97% chemical purity, more preferably about 98% chemical purity, and most preferably about 100% chemical purity.

The term “chemical purity,” as used herein, means percentage of R¹SO₂NHR².HCl in a sample. A sample of R¹SO₂NHR²HCl, may contain, for example, acetic acid, ethanol, ethyl acetate, isopropyl acetate, isopropyl ether, methanol, n-propanol, pyridine, pyridine hydrochloride, water, 4-aminophenol, 3,4-bis(4-hydroxyanilino-6-((4-hydroxyphenyl)imino-2,4-cyclohexadien-1-one of varying geometric purity, 2-chloro-3-nitropyridine or a regioisomer thereof, 2,6-di-tert-butylphenol, 4-((3-nitro-2-pyridinyl)oxy)aniline, p-methoxybenzenesulfonyl chloride, 4-((3-(((4-methoxyphenyl)sulfonyl)amino)pyridin-2-yl)amino)phenyl 4-methoxybenzenesulfonate or a mixture thereof.

Unless stated otherwise, percentages herein are weight/weight (w/w) percentages.

The term “mixture,” as used herein, means a combination of two or more than two substances. For mixtures comprising or consisting essentially of R¹SO₂NHR² and solvent, the R¹SO₂NHR² may be completely soluble, partially soluble, or essentially insoluble in the solvent.

It is meant to be understood that solvent molecules from solvated R¹SO₂NHR².HCl may be used as solvent for preparation of crystalline R¹SO₂NHR².HCl.

The term “solvate,” as used herein, means including a solvent such as acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethyl-acetamide, N,N-dimethylformamide, formamide, formic acid, heptane, hexane, isopropanol, methanol, 1-methyl-2-pyrrolidinone, mesitylene, nitromethane, polyethylene glycol, propanol, 2-propanone, pyridine, tetrahydrofuran, toluene, water, xylene, or a mixture thereof.

Mixtures comprising R¹SO₂NHR²HCl and solvent are mixtures made with crude R¹SO₂NHR²HCl, in which the impurity may be completely soluble, partially soluble, or essentially insoluble in the solvent. The level of chemical impurity may be lowered before or during isolation of crystalline R¹SO₂.NHR HCl by means such as recrystallization, reverse-phase high performance liquid chromatography, ion exchange column chromatography, trituration, extraction, filtration distillation, or a combination thereof.

Causing crystalline R¹SO₂NHR².HCl to exist in a mixture comprising R¹SO₂NHR²and solvent, wherein the R¹SO₂NHR² is completely soluble in the solvent, is known as nucleation.

Nucleation may be made to occur by means such as solvent removal, temperature change, solvent-miscible anti-solvent addition, solvent-immiscible anti-solvent addition, seed crystal addition of a particular crystalline R¹SO₂NHR², chafing or scratching the interior of the container, preferably a glass container, in which nucleation is meant to occur with an implement such as a glass rod or a glass bead or beads, or a combination of the foregoing.

For the practice of this invention, nucleation may be followed by crystal growth, accompanied by crystal growth, or followed and accompanied by crystal growth during which the percentage of a particular crystalline R¹SO₂NHR².HCl increases and as a result of which, crystalline R¹SO₂NHR².HCl having substantial crystalline purity develops.

The term “solvent,” as used herein, means a substance, preferably a liquid or a miscible, partially miscible or essentially immiscible mixture of two or more than two liquids, which is capable of completely dissolving, partially dissolving, dispersing or partially dispersing another substance, preferably a solid or a mixture of solids.

The term “miscible,” as used herein, means capable of combining without separation of phases.

The term “anti-solvent,” as used herein, means a solvent in which R¹SO₂NHR².HCl is essentially insoluble at a particular temperature and concentration.

It is meant to be understood that, because many solvents and anti-solvents contain impurities, the level of impurities in solvents and anti-solvents for the practice of this invention, if present, are at a low enough percentage that they do not interfere with the intended use of the solvent in which they are present.

The term “seed crystal,” as used herein, means crystalline R¹SO₂NHR².HCl having mass. It is meant to be understood that such a crystal may be small enough to be airborne or invisible to the eye.

The term “isolating” or “isolation,” as used herein, means separating R¹SO₂NHR².HCl and impurity, wherein the impurity may be solvent, anti-solvent, a solid 10 or a mixture thereof. Isolation of R¹SO₂NHR².HCl is typically accomplished by means such as centrifugation, filtration with or without vacuum, filtration under positive pressure, distillation, evaporation or a combination thereof.

Crystalline R¹SO₂NHR².HCl may also be made by the procedures described hereinbelow.

### EXAMPLE 1

A mixture of 2-chloro-3-nitropyridine (2C3NP, 138.1 Kg), 4-aminophenol (2.5-3 equivalents) and N,N-dimethylformamide (DMF, 4.8 mL/g 2C3NP) was stirred until homogeneous, heated at 50° C. during which an exotherm raised the solution temperature to 70° C., warmed to 80-85° C., stirred until no 2-chloro-3-nitropyridine remained, cooled to 30° C., treated with water (10.6 mL/g 2C3NP) to precipitate product, then with acetic acid (1.2 mL/g 2C3NP), then with ethyl acetate (0.5 mL/g 2C3NP), cooled to 5° C., stirred for 2 hours and filtered. The filtrant was washed sequentially with distilled water (1.6 mL/g 2C3NP), cold ethanol (1.2 mL/g 2C3NP) and cold isopropyl ether (1.2 mL/g 2C3NP), and dried under vacuum.

In a preferred embodiment of this process, 4-aminophenol (1 equivalent) was used with 4-methylmorpholine (1.5 equivalents) in either methanol or DMF, and precipitation was accomplished with 10% aqueous acetic acid.

### EXAMPLE 2

A mixture of EXAMPLE 1 (41.05 Kg) and ammonium formate (5 equivalents), with or without 2,6-di-tert-butylphenol antioxidant, was treated with a mixture of 50% wet 5% palladium hydroxide on carbon (7% by weight per weight of EXAMPLE 1), in DMF (6 mL/g catalyst) then DMF (total DMF volume: 5 mL/g EXAMPLE 1) first with moderate agitation to control an exotherm (typically peaking at 85° C.) then with increased agitation for 1 hour (incomplete reactions were treated with additional catalyst/DMF mixture), cooled to 10° C., and filtered. The filtrant was washed with DMF (0.4 mL/g EXAMPLE 1), and the filtrate was added to water (29.4 mL/g EXAMPLE 1) at 10° C. to precipitate a solid which was filtered, washed with water (7.5 mL/g EXAMPLE 1), partially dried under a nitrogen stream, and further dried under vacuum at 50° C. to about 0.5% moisture.

### EXAMPLE 3

A mixture of EXAMPLE 2 in pyridine (9 mL/g) at 0° C. was treated with a mixture of para-methoxybenzenesulfonyl chloride (1.05 equivalents) in THF (1.4 mL/g) at 0° C. at a rate which kept the reaction temperature below 5° C., warmed to 25° C., stirred for 15 minutes, and concentrated. The concentrate was treated with n-propanol to provide a composition having 9% pyridine in the solvent mixture and to precipitate a solid. The mixture was cooled to 0° C. and filtered. The filtrant and washed with ethyl acetate (5-7 mL/g starting material) and dried at 45° C.

A sample of crystalline R¹SO₂NHR².HCl of this invention for powder diffraction analysis was applied as a thin layer, with no prior grinding, to the analysis well of a Scintag XDS 2000 Diffractometer having the following parameters: x-ray source: Cu-Kα; range: 2.00°-40.00° 2θ; scan rate: 1.00 degree per minute; step size: 0.02°; temperature: about 25° C.; wavelength: 1.54178 Å.

Representative characteristic peak positions in the powder diffraction pattern of crystalline R¹SO₂NHR.HCl of this invention, expressed as degrees relative to 2θ, are, when measured at about 25° C. with Cu-Kα radiation, about 11.7° (1,0,-1), 12.8°(1,1,-1), 13.2° (1,0,1), 14.4° (0,1,2), 15.9° (1,2,-1), 16.7° (1,1,-2), 17.2° (0,2,2), 19.2° (1,2,-2), 21.1° (0,3,2), 21.4° (1,0,-3), 22.7° (0,4,1), 22.9° (0,2,3), 24.2° (1,4,0), 24.7° (1,1,3), 25.5° (2,2,1), 25.6° (0,4,2) or 26.0° (0,3,3). Each peak position is shown with its accompanying Miller index (h,k,l) values.

The term “about” preceding a series of peak positions is meant to include all of the peak positions of the group which the term precedes.

It is meant to be understood that peak heights may vary and will be dependent on variables such as the temperature, size of crystal size or morphology, sample preparation, or sample height in the analysis well of the diffractometer.

It is also meant to be understood that peak positions may vary when measured with different radiation sources. For example, Cu-Kα₁, Mo-Kα, Co-Kα and Fe-Kα radiation, having wavelengths of 1.54060 Å, 0.7107 Å, 1.7902 Å and 1.9373 Å, respectively, may provide peak positions which differ from those measured with Cu-Kα radiation.

While digital outputs from powder x-ray diffractometers may be set to express peak positions to the one-hundredth and one-thousandth of a degree past the decimal, diffractometers are incapable of accurate experimental determination beyond one-tenth of a degree. Accordingly, peak positions reported herein are rounded to one-tenth of a degree past the decimal.

Peak positions may also be expressed with a variability which accounts for differences between powder x-ray diffractometers, and variability between Cu-Kα radiation sources, variability from sample to sample on the same diffractometer, and differences in sample heights in the analysis well. This variability is preferably expressed as about ±0.2°, about ±0.1°, or a combination thereof.

Therapeutic utility of R¹SO₂NHR².HCl is demonstrated in commonly-owned U.S. application Ser. No. 10/857,235, May 28, 2004 and United States Application Ser. No. 60/575,577, May 28, 2004, the specifications of which are hereby incorporated by reference into this application.

R¹SO₂NHR² binds to the colchicine site of tubulin β-subunits and inhibits the polymerization of tubulin. Accordingly, the compound is useful as a drug for treating diseases in a mammal which are caused or exacerbated by polymerization of tubulin. Such diseases include, but are not limited to, cancer and gouty arthritis, wherein cancer includes, but is not limited to, bone marrow dyscrasias, breast (ductal and lobular) cancer, cervical cancer, colon cancer, leukemia, lung (small cell and non-small cell) cancer, lymphoma, melonoma, mouth and tongue cancer, neuroblastoma (including pediatric neuroblastoma), pancreatic cancer, prostate cancer, rectal cancer, renal cancer, sarcoma, stomach cancer, uterine cancer, and cancers resulting from the metastasis of disease from these areas.

The term “mammal,” as used herein, means a particular class of vertebrate, preferably a human.

Compositions comprising crystalline R¹SO₂NHR².HCl may be administered, for example, bucally, ophthalmically, orally, osmotically, parenterally (intramuscularly, intraperintoneally, intrastemally, intravenously, subcutaneously), rectally, topically, transdermally, or vaginally. Ophthalmically administered dosage forms may be administered as, for example, elixirs, emulsions, microemulsions, oinments, solutions, suspensions, or syrups. Orally administered solid dosage forms may be administered as, for example, capsules, dragees, emulsions, granules, pills, powders, solutions, suspensions, tablets, microemulsions, elixirs, syrups, or powders for reconstitution. Osmotically and topically administered dosage forms may be administered as, for example, creams, gels, inhalants, lotions, ointments, pastes, or powders. Parenterally administered dosage forms may be administered, as, for example, aqueous or oleaginous solutions or suspensions. Rectally and vaginally dosage forms may be administered as, for example, creams, gels, lotions, ointments or pastes.

The therapeutically acceptable amount of crystalline R¹SO₂NHR².HCl depends on recipient of treatment, the disease and severity thereof, the composition containing it, time of administration, route of administration, duration of treatment, its potency, its rate of clearance and whether or not another drug is co-administered. The amount of crystalline R¹SO₂NHR².HCl used to make a composition to be administered daily to a patient in a single dose or in divided doses is from about 0.03 to about 200 mg/kg body weight. Single dose compositions contain these amounts or a combination of submultiples thereof.

Crystalline R¹SO₂NHR².HCl may be administered with or without an excipient. Excipients include, but are not limited to, encapsulating materials and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof.

Excipients for preparation of compositions comprising crystalline R¹SO₂NHR².HCl to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl ftimarate, sucrose, surfactants, talc, tragacanth, tetrahydrofurfuryl alcohol, triglycerides, water, and mixtures thereof. Excipients for preparation of compositions made with crystalline R¹SO₂NHR².HCl to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, and mixtures thereof. Excipients for preparation of compositions made with crystalline R¹SO₂NHR².HCl to be administered osmotically include, for example, chlorofluorohydrocarbons, ethanol, water, and mixtures thereof. Excipients for preparation of compositions made with crystalline R¹SO₂NHR².HCl to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for preparation of compositions made with or comprising crystalline R¹SO₂NHR².HCl to be administered rectally or vaginally include, for example, cocoa butter, polyethylene glycol, wax, and mixtures thereof.

The foregoing is meant to be illustrative of the invention and not intended to limit it to the embodiments disclosed herein. Variations and changes obvious to one skilled in the art are intended to be within the scope and nature of the invention as defined in the claims.

What is claimed is:

1. Crystalline N-(2-((4-hydroxyphenyl)amino)pyridin-3-yl)-4-methoxybenzenesulfonamide hydrochloride.

2. N-(2-((4-Hydroxyphenyl)amino)pyridin-3-yl)-4-methoxybenzenesulfonamide hydrochloride Crystalline Form 1 characterized, when measured at about 25° C. with Cu-Kα radiation, by a powder diffraction pattern with at least three peaks having respective 2θvalues of about 11.7°, 12.8°, 13.2°, 14.4°, 15.9°, 16.7°, 17.2°, 19.2°, 21.1°, 21.4°, 22.7°, 22.9°, 24.2°, 24.7°, 25.5°, 25.6°or 26.0°.

3. N-(2-((4-Hydroxyphenyl)amino)pyridin-3-yl)-4-methoxybenzenesulfonamide hydrochloride Crystalline Form 1 characterized, when measured at about 25° C. with Mo-Kα radiation, by respective lattice parameters a, b and c of about 8.4918 Å±0.0009 Å, 16.389 Å±0.0004 Å and 13.358 Å±0.0002 Å, and β of 98.052°.

4. A composition comprising an excipient and a therapeutically acceptable amount of a crystalline N-(2-((4-hydroxyphenyl)amino)pyridin-3-yl)-4-methoxybenzenesulfonamide hydrochloride.

5. A method for treating cancer in a mammal comprising administering a therapeutically effective amount of a crystalline N-(2-((4-hydroxyphenyl)amino)pyridin-3-yl)-4-methoxybenzenesulfonamide hydrochloride.

6. A process for making N-(2-((4-Hydroxyphenyl)amino)pyridin-3-yl)-4-methoxybenzenesulfonamide hydrochloride Crystalline Form 1, said processes comprising:
providing a mixture comprising N-(2-((4-hydroxyphenyl)amino)pyridin-3-yl)-4-methoxybenzenesulfonamide hydrochloride and solvent, wherein said N-(2-((4-hydroxyphenyl)arnino)pyridin-3-yl)-4-methoxybenzenesulfonarnide hydrochloride is completely soluble in said solvent;
causing crystalline N-(2-((4-Hydroxyphenyl)amino)pyridin-3-yl)-4-methoxybenzenesulfonamide hydrochloride Crystalline Form 1 to exist in said mixture, said N-(2-((4-hydroxyphenyl)amino)pyridin-3-yl)-4-methoxybenzenesulfonamide hydrochloride, when isolated and measured at about 25° with Mo-Kα radiation, characterized by respective lattice parameters a, b and c of about a, b and c of about 8.4918 Å±0.0009 Å, 16.389 Å±0.0004 Å and 13.358 Å±0.0002 Å, and β of 98.052°; and
isolating said N-(2-((4-Hydroxyphenyl)amino)pyridin-3-yl)-4-methoxybenzenesulfonamide hydrochloride Crystalline Form 1.
